(19) United States
(12) Patent Application Publication
(10) Pub. No.: US 2002/0192308 A1
(43) Pub. Date: Dec. 19, 2002

(54) Method and composition for controlling weight

(76) Inventor: John Mamana, McLean, VA (US)

Correspondence Address: McGuire Woods, LLP, Suite 1800, 1750 Tysons Boulevard, McLean, VA, 22102, US

(21) Appl. No.: 09/880,084

(22) Filed: Jun. 14, 2001

(51) Int. Cl. A61K 35/78; A61K 33/24; A61K 31/555; A61K 31/404

(52) U.S. Cl. 424/729; 424/760; 424/655; 514/184; 514/419

(57) ABSTRACT

An appetite suppressant and a method for controlling the weight of a person is described. The appetite suppressant is a composition that includes a chromium additive, green tea, and green tea leaf extract containing catechin polyphenols. The method for controlling the weight of a person includes replacing at least one meal per day with a soy meal replacement and taking an appetite suppressant that contains a chromium additive, green tea, and green tea leaf extract. Also described is a weight control kit that includes a soy meal replacement and an appetite suppressant that contains a chromium additive, green tea, and green tea leaf extract.

### FIELD OF THE INVENTION

The present invention is directed to an appetite suppressant and a method for regulating weight. More particularly, the present invention is directed to an appetite suppressant that includes green tea extract, green tea, and a chromium additive. Further, the invention is directed to a method that utilizes the green tea extract, green tea, and a chromium additive containing appetite suppressant in a program that regulates weight.

### SUMMARY OF THE INVENTION

Accordingly, the present invention is directed to an appetite suppressant comprising green tea, green tea leaf extract, and a chromium additive. The chromium additive may be in a form selected from the group consisting of polynicontinate, amino acid chelate, a chloride, and picolinate, and combinations thereof. The appetite suppressant may further comprise 5-hydroxytryptophan and beta-hydroxy beta-methylbutyrate. Preferably, the green tea is in an amount ranging from about 175 mg to about 325 mg and the green tea leaf extract is in an amount ranging from 10 mg to about 90 mg. The green tea leaf extract preferably contains about 50% catechin polyphenols. Most preferably, the green tea is in an amount of about 250 mg. and the green tea leaf extract is in an amount of about 50 mg. Preferably, the chromium additive ranges from about 50 mg to about 200 mg. The appetite suppressant may be in the form of a tablet, capsule, or powder.

Further, the invention includes an appetite suppressant consisting essentially of green tea, green tea leaf extract, a chromium additive, 5-hydroxytryptophan, and betahydroxy beta-methylbutyrate. Preferably, the green tea is in an amount ranging from about 180 mg to about 325 mg and the green tea leaf extract is in an amount ranging from about 10 mg to about 90 mg. Preferably, the chromium additive ranges from about 50 mg to about 200 mg. The appetite suppressant may be in the form of a tablet, capsule or powder. Still further, the present invention includes an appetite suppressant consisting essentially of green tea, green tea leaf extract, a chromium additive, 5-hydroxytryptophan, beta-hydroxy beta-methylbutyrate, potassium glycerophosphate, a calcium additive, a vatinum additive, L-carnitine, L-tartrate, and a cayenne fruit additive. Preferably, the chromium additive is in a form selected from the group consisting of a polynicotinate, an amino acid chelate, a chloride, a picolinate, and combinations thereof. Preferably, the calcium additive is in the form of calcium carbonate. The green tea leaf extract preferably contains catechin polyphenols. Preferably, the green tea is in an amount ranging from about 180 mg to about 325 mg and the green tea leaf extract is in an amount ranging from about 10 mg to about 90 mg. Preferably, the chromium additive ranges from about 50 mg to about 200 mg. The appetite suppressant may be in the form of a tablet, capsule, or powder.

The present invention also includes a weight control kit comprising an appetite suppressant wherein the appetite suppressant contains green tea, green tea leaf extract, and a chromium additive. The kit includes a soy based meal replacement.

Further, the invention includes a method for controlling weight comprising the steps of ingesting an appetite suppressant, wherein the appetite suppressant contains green tea, green tea leaf extract, and a chromium additive, and replacing at least one meal with a soy based meal replacement.

### DETAILED DESCRIPTION OF THE INVENTION

The composition for controlling weight of the present invention contains green tea, green tea leaf extract, and a chromium additive. The composition is an appetite suppressant and helps improve the efficiency of burning fat.

The green tea and the green tea leaf extract contain antioxidant compounds that improve fat burning efficiency. The green tea component is preferably in the form of green tea powder. The green tea is added in an amount effective to improve the efficiency of burning fat in an individual. This range may vary depending on the individual. Preferably, the amount of green tea ranges from about 175 mg to about 325 mg per dose of appetite suppressant. Most preferably, the amount of green tea is about 250 mg per dose.

The green tea leaf extract, like the green tea component, helps improve the fat burning efficiency of an individual. The green tea leaf extract is an extract from green tea. The extraction process includes processes known to one skilled in the art. One method of extraction is an aqueous extraction method that uses a low temperature process. The green tea is spray dried under hot air. Green tea extract droplets containing catechin polyphenols fall out under gravity and are collected. The extract is assayed to a standard amount of about 50% catechin polyphenols using an inert diluent such as maltodextrin.

Preferably, the green tea leaf is extracted such that it contains about 50% catechin polyphenols. The amount of green tea leaf extract and the catechin polyphenols may vary per dose, but should be an amount effective, when combined with the green tea powder, to help improve fat burning in an individual. This range may vary depending on the individual and may depend on an individual's weight, metabolism, and other characteristics of the individual known to those skilled in the art. Preferably, the amount of green tea leaf extract per dose may range from about 10 mg to about 90 mg. Most preferably, the green tea leaf extract per dose ranges from about 35 mg to about 65 mg.

The chromium additive is part of the composition that suppresses the appetite by stabilizing blood sugar and insulin levels. When the chromium is combined with the green tea powder and green tea leaf extract components, the composition places the body in a mode that helps control weight gain in an individual. The chromium additive may be added in any number of forms. The form of chromium may include, but is not limited to a polynicotinate, an amino acid chelate, a chloride, a picolinate, and any combination of these or other similar chromium additives. The amount of chromium additive per dose may range from about 50 mg chromium to about 200 mg chromium.

Other additives maybe included in the composition. For example, 5-hydroxytryptophan (5HTP) and beta-hydroxy beta-methylbutyrate (HMB) may be added either alone or in combination with each other. 5HTP is useful because it is a precursor to serotonin production which helps to elevate the mood of an individual which can in turn decrease the appetite of the individual. In one embodiment, 5HTP is preferably added in an amount up to about 30 mg per dose. In one embodiment of the present invention 5HTP is added in an amount of about 17 mg. Further, an extract of Griffonia, about 15% to about 20%, may be used. Preferably, about 50 mg to about 150 mg of a 15% to about 20% extract of Griffonia may be used in the compositions of the present invention.

HMB typically aids in the development of lean muscle mass. When an individual is burning fat, the development of lean muscle mass is important. In another embodiment, HMB is preferably added in an amount up to about 75 mg per dose. In one embodiment of the present invention HMB is added in an amount of about 50 mg.

The composition may include other additives that provide nutrient support for the body. For example, vitamins, minerals, amino acids may be included in the composition. Other additives may include, but are not limited to, potassium glycerophosphate, calcium additives such as calcium carbonate, vatinum additives, L-carnitine, L-tartrate, and fruit additives such as cayenne fruit additive.

The composition is preferably administered orally in the form of a tablet, capsule or powder. Any pharmaceutically acceptable methods for forming or preparing the tablet, capsule, or powder may be used. When a tablet is the desired form, any pharmaceutically acceptable additional components, binders, and the like may be added to the composition to form the tablet.

The above mentioned preferable ranges for components of the composition are for a single dose of the composition. The number of doses for most individuals is preferably three times a day, and preferably before meals. The number of doses, the time of the doses, and the time period for administering the composition may vary from one patient to the next. Some of the factors in determining the number and time of the doses and the period of administration include the size and weight of the patient, the patients metabolism, the patients response to the composition, and other factors known to one skilled in the art.

In addition to taking the composition of the present invention for controlling weight, a preferable method also includes replacing one meal per day with a low calorie supplement. Reducing the caloric intake allows the composition to help increase fat metabolism and regulate weight in an effective manner. A suitable meal replacement is one that ranges from 200-300 calories. Preferably, the meal replacement is a soy-based consumable. The replacement is preferably in the form of a shake and preferably contains nutrients for the individual. Nutrients may include, but is not limited to, amino acids, vitamins, minerals, and fiber. The replacement may include a flavor such a vanilla, chocolate, strawberry, and the like. In addition to taking the appetite suppressant and meal replacement, it is preferable that the patient restrict their daily diet to 1200-1600 calories per day and regularly exercise.

The method of the present invention may be used to lose weight or maintain a certain weight. Additionally, the composition and meal replacement may be included in a kit that contains a supply of the appetite suppressant and a supply of the meal replacement. Preferably, the kit would contain a supply of meal replacement that corresponds to the supply of the appetite suppressant. The kit could be structured such that it includes a daily, weekly or monthly, yearly supply of appetite suppressants and meal replacements. Instructions and guidelines on the usage and dosage of the appetite suppressant and meal replacement may also be included. Similarly, a chart for tracking the individuals' progress may be included in the kit. The chart may track information such as weight, caloric intake, dosage of appetite suppressant, and the like. The chart may track the information over time periods such as daily, weekly, monthly, or yearly. Preferably the method is followed under the supervision an appropriate health care professional.

The present invention is illustrated in the following example. The example is provided for illustration purposes and should not be construed as limiting the scope of the present invention.

### EXAMPLE

Overweight individuals were given an appetite suppressant in the form of a tablet three times a day before meals. The appetite suppressant contained:

250 mg of green tea powder;

50 mg of green tea leaf extract containing 50% catechin polyphenols;

80 mg of chromium as polynicotinate, amino acid chelate, chloride, and picolinate;

17 mg of 5-HTP;

50 mg of HMB;

200 mg of calcium carbonate;

25 mg of potassium glycerophosphate;

10 mg of L-Carnitine and L-Tartrate;

10 mg of Cayenne fruit; and

10 mg of vanadium in the form of vandyl sulfate.

The individuals replaced one meal per day with a chocolate or vanilla soy shake containing 220 calories, restricted their diet to 1200-1600 calories per day, and exercised regularly. 156 individuals participated in the program for three months. Over the three month period, the individuals lost at least 10% of their body weight. The individuals reported that the appetite suppressant was effective curbing their hunger.

It will be readily understood by those persons skilled in the art that the present invention is susceptible to broad utility and application. Many embodiments and adaptations of the present invention other than those herein described, as well as many variations, modifications and equivalent arrangement, will be apparent from or reasonably suggested by the present invention and the foregoing description without departing from the substance or scope of the present invention.

Accordingly, while the present invention has been described in detail in relation to its preferred embodiment, it is to be understood that this disclosure is only illustrative and exemplary of the present invention and is made merely for purposes of providing a full and enabling disclosure of the invention. The foregoing disclosure is not intended to be construed to limit the present invention or otherwise exclude any other embodiments, adaptations, variations, modifications or equivalent arrangements, the present invention being limited only by the claims and the equivalents thereof.

What is claimed is:

1. An appetite suppressant comprising:
green tea;
green tea leaf extract; and
a chromium additive.

2. The appetite suppressant of claim 1 wherein the chromium additive is in a form selected from the group consisting of polynicontinate, amino acid chelate, a chloride, and picolinate, and combinations thereof.

3. The appetite suppressant of claim 1 further comprising:
5-hydroxytryptophan; and
beta-hydroxy beta-methylbutyrate.

4. The appetite suppressant of claim 1 wherein the green tea is in an amount ranging from about 175 mg to about 325 mg.

5. The appetite suppressant of claim 1 wherein the green tea leaf extract is in an amount ranging from 10 mg to about 90 mg.

6. The appetite suppressant of claim 1 wherein the green tea leaf extract contains about 50% catechin polyphenols.

7. The appetite suppressant of claim 1 wherein the green tea is in an amount of about 250 mg and the green tea leaf extract is in an amount of about 50 mg.

8. The appetite suppressant of claim 1 wherein the appetite suppressant is in a form selected from the group consisting of a tablet, a powder, and a capsule.

9. The appetite suppressant of claim 1 wherein the chromium ranges from about 50 mg to about 200 mg.

10. An appetite suppressant consisting of essentially of:
green tea
green tea leaf extract containing catechin polyphenols;
a chromium additive;
5-hydroxytryptophan; and
beta-hydroxy beta-methylbutyrate.

11. The appetite suppressant of claim 10 wherein:
green tea is in an amount ranging from about 180 mg to about 325 mg; and
green tea leaf extract is in an amount ranging from about 10 mg to about 90 mg.

12. The appetite suppressant of claim 10 wherein the appetite suppressant is in a form selected from the group consisting of a tablet, a powder, and a capsule.

13. The appetite suppressant of claim 10 wherein the chromium ranges from about 50 mg to about 200 mg.

14. The appetite suppressant consisting essentially of:
green tea;
green tea leaf extract containing catechin polyphenols;
a chromium additive;
5-hydroxytryptophan;
beta-hydroxy beta-methylbutyrate;
potassium glycerophosphate;
a calcium additive;
a vatinum additive;
L-carnitine;
L-tartrate; and
a cayenne fruit additive.

15. The appetite suppressant of claim 14 wherein the chromium additive is in a form selected from the group consisting of a polynicotinate, an amino acid chelate, a chloride, a picolinate, and combinations thereof.

16. The appetite suppressant of claim 14 wherein the calcium additive in the form of calcium carbonate.

17. The appetite suppressant of claim 14 wherein the green tea leaf extract contains about 50% catechin polyphenols.

18. The appetite suppressant of claim 14 wherein the green tea is in an amount ranging from about 180 mg to about 325 mg and the green tea leaf extract is in an amount ranging from 10 mg to about 90 mg.

19. The appetite suppressant of claim 14 wherein the appetite suppressant is in a form selected from the group consisting of a tablet, a powder, and a capsule.

20. The appetite suppressant of claim 14 wherein the appetite suppressant is in the form of a powder.

21. A weight control kit comprising:
an appetite suppressant wherein the appetite suppressant contains green tea, green tea leaf extract, and a chromium additive; and
a soy based meal replacement.

22. A method for controlling weight comprising the steps of:
ingesting an appetite suppressant, wherein the appetite suppressant contains green tea, green tea leaf extract, and a chromium additive; and
replacing at least one meal with a soy based meal replacement.
